SEQUENCE LISTING (7 sequences)

SEQ ID NO: 1 - protein, 53 aa, Artificial Sequence
  Synthetic peptide
NSDSECPLSHDGYCLHDGVCMYIEALDKYACNCVVGYIGERCQYRDLKWWELR

SEQ ID NO: 2 - protein, 55 aa, Artificial Sequence
  Synthetic peptide
VVSHFNDCPLSHDGYCLHDGVCMYIEALDKYACNCVVGYIGERCQYRDLKWWELR

SEQ ID NO: 3 - protein, 48 aa, Artificial Sequence
  Synthetic peptide
NSDSECPLSHDGYCLNDGVCMYTEALDEYACNCVVGYIGERCQYYDLL

SEQ ID NO: 4 - protein, 48 aa, Artificial Sequence
  Synthetic peptide
NSDSECPLSHDGYCLNDGVCMYTEAKDEYACNCVVGYIGERCQYYDLL

SEQ ID NO: 5 - protein, 22 aa, Artificial Sequence
  Synthetic peptide
TYLVEKPNLSSKRSELSQLSMY

SEQ ID NO: 6 - protein, 9 aa, Artificial Sequence
  Synthetic peptide
TYLVEKPNL

SEQ ID NO: 7 - protein, 5 aa, Artificial Sequence
  Synthetic peptide
QLSMY

(19) United States
(12) Patent Application Publication
(10) Pub. No.: US 2020/0369737 A1
(43) Pub. Date: Nov. 26, 2020

(54) DOMINANT NEGATIVE LIGAND CHIMERIC ANTIGEN RECEPTOR SYSTEMS

(71) Applicant: Daniel J. Monticello, The Woodlands, TX (US)

(72) Inventor: Daniel J. Monticello, The Woodlands, TX (US)

(21) Appl. No.: 16/861,311

(22) Filed: Apr. 29, 2020

Related U.S. Application Data
(63) Continuation of application No. PCT/US18/59198, filed on Nov. 5, 2018.
(60) Provisional application No. 62/582,109, filed on Nov. 6, 2017.

(51) Int. Cl. C07K 14/47; A61K 35/17; C07K 14/65; C07K 14/575; C07K 14/61; C07K 14/725; C12N 7/00; C07K 14/005; A61K 39/165; C07K 16/18; C07K 14/705; C07K 14/485

(52) U.S. Cl. CPC C07K 14/4748; A61K 38/00; C07K 14/65; C07K 14/57554; C07K 14/61; C07K 14/7051; C12N 7/00; C07K 14/005; A61K 39/165; C07K 16/18; C07K 14/70521; C07K 14/70578; C07K 14/485; C07K 2319/03; C07K 2319/02; C07K 2319/30; C07K 2319/33; A61K 35/17

(57) ABSTRACT

The invention provides modified T-cell receptors referred to herein as “dominant negative ligand-chimeric antigen receptors” (DNL-CARS). The present invention also provides T-cells expressing DNL-CARs such T cells also referred to herein as “DNL-CAR-expressing T cells” or “DNL-CAR T cells. Also provided are “tagged-DNL/CAR-T systems” that direct CAR-T cells to tumor cells previously complexed to the DNL-Tag fusion. Also provided are tagged-DNL-antigen fusion proteins wherein the antigen portion of the fusion proteins recruits the patient's own immune system to neutralize cells tagged with the tagged DNL portion of the fusion protein.

### RELATED APPLICATIONS

This application is a continuation of International Application No. PCT/US18/59198, which designated the United States and was filed on Nov. 5, 2018, published in English, which claims the benefit of U.S. Provisional Application No. 62/582,109, filed on Nov. 6, 2017. The entire teachings of the above applications are incorporated herein by reference.

### BACKGROUND OF THE INVENTION

Chimeric Antigen Receptor T cell therapy is an example of cellular immunotherapy, in which the body's own immunological system is directed against abnormal (tumor) cells (Finn O J 2012. Annals in Oncology 23(8): viii6-viii9). Chimeric antigen receptor-expressing T cells (CAR-T) cells were originally developed using the α and ζ (the endodomain) of the T-cell receptor (TCR) and replacing the native TCR ectodomain with a single chain variable region (scFv) of an antibody with affinity for a particular target on a tumor cell (Dotti, G. 2013. Immunological Reviews 257:107-126). These cells bind to tumor cells expressing the target antigen on their surface, which activates the significant tumor killing capabilities of the T cells (Dotti, G. 2013 Immunological Reviews 257:107-126).

CAR T-cell therapy represents a new and potentially powerful approach to controlling and eliminating cancer cells from the body, and there are several versions of this approach now in clinical trials (Ledford H 2014. T-cell therapy extends cancer survival to years. Nature 516:156). Despite the promise of this technology there are several disadvantages of this approach, including the difficulties with selecting an appropriate target antigen on the tumor cell, and cross reactivity (“off target events”) with other non-target cells (Morgan R A et al. 2010 Mol Ther 18(4)843-851).

There have been attempts to address these difficulties. One approach is to incorporate more than one binding domain in the ectodomain portion of the chimeric antigen receptor (CAR). One such tandem CAR (tanCAR) incorporates extracellular scFv domains that engage two targets, HER2 and CD19, to improve the specificity of the T cells (Grada Z. et al. 2013 Molecular Therapy—Nucleic Acids 2:e105). Another antibody-based approach is to target an inactive variant of Epidermal Growth Factor Receptor (EGFR vIII) that is often expressed on tumor cells (Congdon K L, et al. (2014 Neuro-Oncology 16: viii20-viii25).

Another approach to produce “CAR-like” expressing T-cells is to use proteins that take advantage of the intrinsic binding capabilities of a ligand (such as Epidermal Growth factor, EGF) for its target receptor (EGF Receptor, EGFR). This substitutes the antibody:antigen interaction of conventional CARs with the ligand:receptor interaction in order to direct T cells to the target tumor cells that express the target receptors. Davies et al. (Davies M D et al. 2012. Mol Med 18:565-576) used this approach with a bi-functional ligand that could bind to EGFR and HER2/HER3 dimers. This approach has shown some pre-clinical promise (Van Der Stegen, S J, et al. 2013. J Immunology 191:4589-4598). Unfortunately, the ligand portion of the TCR is still an agonist and can potentially stimulate receptor activation and was very limited in the range of its interactions.

Dominant Negative Ligands have been developed to take advantage of the ligand:receptor interaction, but without the potential activation of the receptors and stimulation of cellular growth. DNL have been reported that antagonize activation of human Growth Hormone Receptor (hGHR) and the Prolactin Receptor. A Pan-HER DNL has been reported (U.S. Pat. No. 7,557,181) that binds to all the ligand-dependent members of the HER family and inhibits activation of all the receptors, including HER2. An IGF-I derived DNL has also been reported (WO2008005985).

The present invention overcomes the drawbacks of the prior art and provides dominant negative ligand chimeric antigen receptor-expressing T lymphocytes (“DNL-CAR T cells”). DNL-CAR T cells are preferably human T cells that have been genetically modified to express a modified T cell receptor comprising a dominant negative ligand (DNL) that is able to recognize particular receptors (Ligand Binding Domains) on the surface of target cells. This is accomplished by fusing a TCR endodomain with a new ectodomain derived from a dominant negative ligand to provide DNL-CAR which is optionally expressed in suitable T cells. DNL-CAR T cells have significant advantages for cancer immunotherapy, including, but not limited to, their ability to recognize and kill tumor cells independently of the major histocompatibility mechanisms.

Another application is to target a CAR-T cell, which is not a DNL-CAR T cell, to a “tag” attached (or fused) to a DNL (i.e. a tagged-DNL). An example of such a tag is polyethylene glycol (PEG). In this way, the DNL aspect of the tag-DNL fusions complex with cells expressing the cognate receptor for the DNL aspect of the tagged-DNL, which is followed by CAR-T therapy aimed at binding the tag (e.g. PEG) on the tagged-DNL. This is possible because of the un-expected observation that the PEGylated DNL are not internalized after binding to their cognate receptors, unlike authentic receptor ligands (agonists).

### SUMMARY OF THE INVENTION

The invention provides modified T-cell receptors referred to herein as “dominant negative ligand-chimeric antigen receptors” (DNL-CARS). The present invention also provides T-cells expressing DNL-CARS such T cells also referred to herein as “DNL-CAR-expressing T cells” or “DNL-CAR T cells”. The invention provides nucleic acids encoding DNL-CAR polypeptides and expression vectors useful for transforming T cells to express DNL-CARS of the invention. The invention also provides pharmaceutical formulations comprising DNL-CARS and DNL-CAR T cells, and methods of using such formulations in the treatment of disease. The invention also provides targets for CAR-T cells based on molecular “tags” on the DNL that direct the CAR-T cells to the tumor cells. Also provided are tagged-DNL-antigen fusion proteins wherein the antigen portion of the fusion proteins recruits the patient's own immune system to neutralize cells tagged with the tagged DNL portion of the fusion protein.

### BRIEF DESCRIPTION OF THE DRAWINGS

The following drawings form part of the present specification and are included to further demonstrate certain aspects of the present invention.

FIG. 1 is a schematic of a plasmid map encoding a DNL-CAR of the invention.

FIG. 2 shows in vitro ligand binding affinities to the cognate receptors of the Pan-HER DNL (“B5-3M2”) tagged with biotin. Biotin-labeled ligands at the concentrations indicated were added to wells with receptor extracellular domain/Fc chimera captured on anti-Fc coated plates. Results represent the mean of 2 experiments performed in duplicate. The Kd values calculated from the curves were 0.7 nM for EGF-EGFR, 5 nM for B5-3M2-EGFR, 6 nM for B5-3M2-HER3 and 4 nM for B5-3M2-HER4.

FIG. 3 illustrates the PEGylation of the Pan-HER DNL. Gel A shows the image of the gel strained for protein. Gel B is the same gel, stained also with barium Iodide, which reacts with the PEG. This illustrates that protein is readily PEGylated.

FIG. 4 shows the inhibition of T47D proliferation in the presence of the agonist A1B1 with PEGylated B5-3M2 and PEGylated B5 2M3.

FIG. 5 shows the inhibition of ligand internalization by the tagged (PEGylated) DNL.

FIG. 6 illustrates some potential uses of the DNL-CAR and Tagged-DNL/CAR-T systems. Panels A and B refer to the DNL-CAR system, and panels C and D refer to the tagged-DNL-CAR T system.

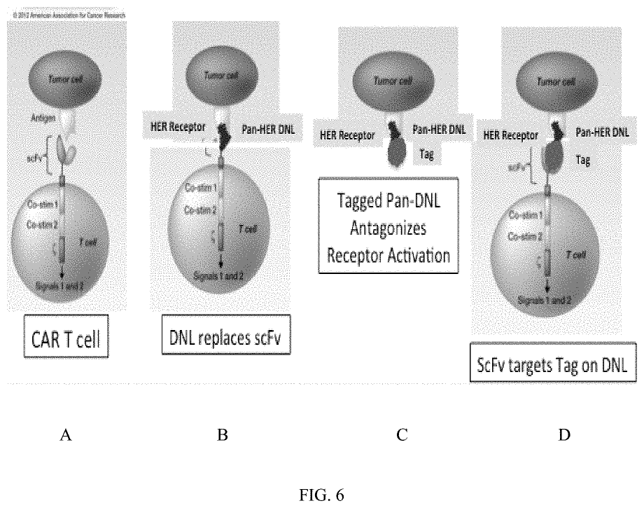

### DETAILED DESCRIPTION OF THE INVENTION

As used herein the term “dominant negative ligand” (DNL) is used to describe that type of ligand, which, when altered or modified to differ from the native or wild-type ligand in any respect, results in a ligand that retains binding affinity for a wild-type binding receptor (e.g. a receptor) but inhibits the function or signaling of the wild-type binding partner. As used herein the term “ligand” is used to designate a polypeptide based molecule capable of specific binding to a receptor as herein defined. The definition includes any native ligand for a receptor or any region or derivative thereof retaining at least a qualitative receptor binding ability. Specifically excluded from this definition are antibodies to a receptor and noncovalent conjugates of an antibody and an antigen for that antibody. The terms “native ligand” and “wild-type ligand” are used interchangeably and refer to an amino acid sequence of a ligand occurring in nature (“native sequence ligand”), including mature, pre-pro and pro forms of such ligands, purified from natural source, chemically synthesized or recombinantly produced. Native ligands that can activate receptors are well known in the art or can be prepared by art known methods.

There are many ligands: receptor interactions that have been implicated in the progression of different cancers (Citri A and Y Yardin 2006. Molecular Cell Biology 7, 505-516). All of these are candidates for the development of dominant negative ligands using techniques previously described in WO2008005992. There are other cellular and extracellular Ligand Binding Domains that have not been characterized as receptors per se but interact with extracellular ligands and can be considered receptors in this context.

The method of making a DNL involves ablating at least one binding domain on a multivalent ligand and optionally using a binding optimization method to enhance binding at a second site on the specific target receptor. Re-engineering of a ligand to make it a DNL comprises making one or more modifications to one or more features at a first receptor binding surface of the ligand to disrupt binding of the ligand to a first target receptor domain, and (if necessary) making one or more modifications to one or more features at a second receptor binding surface of the ligand to enhance binding of the ligand to a second target receptor domain. Ligands are selected from either known receptor ligands or a polypeptide sequence that functions as a ligand. The ligands, once having undergone domain binding optimization, are then assayed for their ability to inhibit a biological activity in one or more cell lines wherein the biological activity is selected from the group consisting of a receptor-mediated pathology, receptor-mediated cell signaling, cell growth, cell proliferation and tumor growth.

Desirably, the inhibited biological activity is receptor-mediated cell signaling. This inhibition of receptor-mediated cell signaling may result in ablation of downstream signaling by a receptor and this effect can be determined by measuring altered phosphorylation states of one or more proteins. One method to evaluate the efficacy of the DNL approach to the inhibition of tyrosine kinase receptor-mediated cell signaling is using autophosphorylation assays or gene expression assays.

This technique has been used to produce several DNL, including Pegvisomant, a PEGylated variant of Human Growth Hormone (hGH) that binds to its cognate receptor, blocking activation by the normal ligand. This compound is FDA approved for the treatment of acromegaly. Another example is a variant of prolactin, which binds to the Prolactin Receptor (PRLR). However, these DNL only interact with a single receptor type (e.g. hGF Receptor) on the surface of the target cell.

The term “chimeric antigen receptors (CARs),” as used herein, may refer to artificial T-cell receptors, chimeric T-cell receptors, or chimeric immunoreceptors, for example, and encompass engineered receptors that graft an artificial specificity such as a DNL onto a particular immune effector cell. Preferably, CARs of the invention direct specificity of the cell to a tumor associated Ligand Binding Domain. Preferably, CARs comprise an intracellular activation domain (also referred to herein as “endodomain”), a transmembrane domain, and an extracellular domain comprising a Ligand Binding Domain-specific DNL (also referred to herein as an “ectodomain”, or “DNL extracellular domain”). The CAR may also include a linker region to properly position the DNL relative to the target Ligand Binding Domain, and a hinge region to couple the endo- and ecto-domains. Preferably, DNL-CARS of the invention comprise domains for additional co-stimulatory signaling, such as CD3-zeta (CD3ζ), FcR, CD27, CD28, CD137, DAP10, and/or OX40. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

The term “T-cell receptor (TCR)” as used herein refers to a protein receptor on T cells that is composed of a heterodimer of an alpha (a) and beta (β) chain, although in some cells the TCR consists of gamma and delta (γ/δ) chains. In embodiments of the invention, the TCR may be modified on any cell comprising a TCR, including a helper T cell, a cytotoxic T cell, a memory T cell, regulatory T cell, natural killer T cell, and gamma delta T cell, for example.

The terms “tumor-associated antigen” (TAA) and “cancer cell antigen” (CA) are used interchangeably herein. In each case, the terms refer to proteins, glycoproteins carbohydrates or other molecules that are specifically or preferentially expressed by cancer cells, have antigenic and potentially ligand binding domains.

As used herein, the term “Ligand Binding Domain (“LBD”) is a molecule or region of a molecule capable of being bound by any DNL or tagged DNL in accordance with the invention.

As used herein, the term “tagged-DNL” refers to a DNL molecule that is covalently linked to a second molecule (which is the tag), wherein the tag is not a CAR, and wherein a CAR has specificity for the tag and can bind the tag.

As used herein, the term “tagged-DNL/CAR-T system” refers to two separate entities used in combination to treat a cancer: a “tagged DNL” molecule which is not covalently linked to a CAR, and a “CAR-T” T cell that has specificity for, and is capable of binding to, the tag of the “tagged DNL”.

The term “tagged-DNL-antigenic fusion protein” refers to a DNL molecule that is covalently linked to a second molecule (which is the tag) and wherein the DNL is further fused to a peptide or protein that is an antigen. Preferably the tag is not a CAR. “Antigen”, “antigenic” “antigenic protein, “antigenic peptide”, antigenic epitope, “antigenic determinant” all used here as being a sequence of peptidic or glycopeptidic nature that is capable of inducing an immune response in a patient to which it is administered. Accordingly, an antigen may be a protein or part of a protein (polypeptide) or alternatively a small peptide potentially corresponding to an epitope. An “epitope” is the part of an antigen that is recognized by an antibody or by a lymphocyte receptor, such as a T cell receptor. A (linear) epitope is generally constituted by a sequence of from 7 to 15 amino acids. An antigen within the scope of the invention may be constituted by an epitope, may comprise an epitope or may be an antigenic protein. Preferably, the antigen is derived from a protein or peptide used to vaccinate a host and to which the host has developed an immune response. For example, common protein antigens used to vaccinate human hosts include, but are not limited to immunogenic protein fragments from: diphtheria, tetanus, pertussis, polio (IPV), measles, mumps, rubella, chickenpox Hepatitis A, b and H, influenzae and pneumococcal. Other antigens suitable for use in a tagged-DNL-antigen fusion protein of the invention include those that a patient, such as a human patient, may have a non-life threatening exaggerated immune response to. For example, the allergenic proteins or portions thereof from cat dander (e.g. Fel d 1), house dust mites (e.g. Der p 1 and Der p 2), pollens originating from trees, grasses and weeds and molds.

The intracellular signaling domain of the chimeric receptor of the invention is responsible for activation of at least one of the normal effector functions of the immune cell in which the chimeric receptor has been placed. The term “effector function” refers to a specialized function of a differentiated cell. Effector function of a T cell, for example, may be cytolytic activity or helper activity including the secretion of cytokines. Effector function in a naive, memory, or memory-type T cell includes antigen-dependent proliferation. Thus the term “intracellular signaling domain” refers to the portion of a protein that transduces the effector function signal and directs the cell to perform a specialized function.

Preferably, the intracellular signaling domain of a polypeptide expressed by a T lymphocyte described herein is or comprises an intracellular domain of a protein that is normally expressed on the surface of T cells and which triggers activation and/or proliferation of the T cells. While usually the entire intracellular signaling domain will be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term intracellular signaling domain is thus meant to include any truncated portion of the intracellular signaling domain sufficient to transduce the effector function signal. Examples include the zeta chain of the T-cell receptor or any of its homologs (e.g., eta, delta, gamma, or epsilon), MB 1 chain, B29, Fc RIII, Fc RI, and combinations of signaling molecules, such as CD3ζ and CD28, CD27, 4-1BB, DAP-10, OX40, and combinations thereof, as well as other similar molecules and fragments. Intracellular signaling portions of other members of the families of activating proteins can be used, such as FcyRIII and FcsRI. Preferably the intracellular domain is the Human CD3ζ intracellular domain.

The Ligand Binding Domain-specific DNL extracellular domain of the DNL-CAR, or the tag-specific ScFV of the CAR of the tagged-DNL/CAR T system, and the intracellular signaling-domain may be linked by a transmembrane domain, such as the human IgG₄Fc hinge and Fc regions. Alternatives include the human CD4 transmembrane domain, the human CD28 transmembrane domain, the transmembrane human CD3t domain, or a cysteine mutated human CD3ζ domain, or other transmembrane domains from other human transmembrane signaling proteins, such as CD16 and CD8 and erythropoietin receptor.

Preferably a DNL-CAR or a CAR of the tagged-DNL/CAR-T system is encoded by a nucleic acid comprising a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, and 4-1BB (CD137). In addition to a primary signal initiated by CD3ζ, an additional signal provided by a human costimulatory receptor inserted in a human CAR is important for full activation of T cells and could help improve in vivo persistence and the therapeutic success of the adoptive immunotherapy.

The invention also provides isolated nucleic acid segments and expression cassettes incorporating DNA sequences that encode the DNL-CAR of the invention, or that encode the CAR or tagged-DNL used in the tagged-DNL/CAR-T system of the invention. Vectors of the present invention are designed, primarily, to deliver desired genes to immune cells, preferably T cells under the control of regulated eukaryotic promoters, for example, MNDU3 promoter, CMV promoter, EF 1 alpha promoter, or Ubiquitin promoter. Also, the vectors may contain a selectable marker, if for no other reason, to facilitate their manipulation in vitro. In other embodiments, the DNL-CAR of the invention, or the CAR or tagged-DNL used in the tagged-DNL/CAR-T system of the invention, can be expressed from mRNA in vitro transcribed from a DNA template.

DNL-CAR of the invention, or the CAR and tagged-DNL used in the tagged-DNL/CAR-T system of the invention, are preferably recombinant and are distinguished by their ability to both bind Ligand Binding Domain and transduce activation signals via immunoreceptor activation motifs (ITAM's) present in their cytoplasmic tails.

The DNL-CAR, or the tagged-DNL and CAR used in the tagged-DNL/CAR-T system of the invention, may be co-expressed with a membrane-bound cytokine to improve persistence when there is a low amount of tumor-associated Ligand Binding Domain. For example, DNL-CAR can be co-expressed with membrane-bound IL-15.

In constructing the DNL-CAR, or the tagged-DNL and CAR used in the tagged-DNL/CAR-T system provided herein, human sequences may be combined with non-human sequences. For example, a polypeptide comprising human extracellular and intracellular domain amino acid sequences may comprise a transmembrane domain from a non-human species; e.g., may comprise a murine transmembrane domain. DNL-CAR of the invention and CAR used in the tagged-DNL/CAR-T system of the invention may comprise the human amino acid sequences for the extracellular and intracellular domains, and comprises a transmembrane domain derived from a non-human species.

The Ligand Binding Domain to which the extracellular (DNL) domain of the DNL-CARS or the DNL domain of a tagged-DNL provided herein binds/recognizes can be any Ligand Binding Domain of interest, e.g., can be a Ligand Binding Domain on or concentrated near a tumor cell. The tumor cell may be, e.g., a cell in a solid tumor, or cell of a non-solid tumor, e.g., a cell of a blood cancer. The Ligand Binding Domain can be any Ligand Binding Domain that is expressed on or near a cell of any tumor or cancer type, e.g., cells of a lymphoma, a lung cancer, a breast cancer, a prostate cancer, an adrenocortical carcinoma, a thyroid carcinoma, a nasopharyngeal carcinoma, a melanoma, e.g., a malignant melanoma, a skin carcinoma, a colorectal carcinoma, a desmoid tumor, a desmoplastic small round cell tumor, an endocrine tumor, an Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipoma, or the like. In more specific embodiments, said lymphoma can be chronic lymphocytic leukemia (small lymphocytic lymphoma), B-cell prolymphocytic leukemia, lymphoplasmacytic lymphoma, Waldenstrom macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, or a non-Hodgkin lymphoma. Ligand Binding Domains specific to certain cancers, as well as methods for identifying such Ligand Binding Domains, are known in the art.

Preferably, the Ligand Binding Domain recognized by the extracellular domain of a polypeptide described herein is also a tumor-associated antigen (TAA) or a tumor-specific antigen (TSA). In various specific embodiments, the tumor-associated antigen or tumor-specific antigen is, without limitation, EGFR, HER2, HER3, HER4, IGF-IR, other tyrosine kinase receptors, other G-protein coupled receptors, prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD19, CD22, CD27, CD30, CD34, CD45, CD70, CD99, CD117, EGFRvIII (epidermal growth factor variant III), mesothelin, PAP (prostatic acid phosphatase), prostein, TARP (T cell receptor gamma alternate reading frame protein), Trp-p8, STEAP1 (six-transmembrane epithelial antigen of the prostate 1), chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-1), myo-Dl, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, or an abnormal p53 protein.

The TAA or TSA recognized by the DNL domain of the DNL-CARS or by the DNL domain of a tagged-DNL described herein may be a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXB1, SPA17, SSX, SYCP1, or TPTE.

The TAA or TSA recognized by the extracellular DNL domain of a DNL-CAR or by the DNL domain of a tagged-DNL described herein may preferably be a carbohydrate or ganglioside, e.g., fuc-GM1, GM2 (oncofetal antigen-immunogenic-1; OFA-I-1); GD2 (OFA-I-2), GM3, GD3, and the like.

The TAA or TSA recognized by the extracellular DNL domain of a polypeptide or by the DNL domain of a tagged-DNL described herein may preferably be alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-A11, hsp70-2, KIAAO205, Mart2, Mum-1, 2, and 3, neo-PAP, myosin class I, OS-9, pml-RARa fusion protein, PTPR, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gplOO (Pmel 17), tyrosinase, TRP-1, TRP-2, MAGE-1, MAGE-3, RAGE, GAGE-1, GAGE-2, pl5(58), RAGE, SCP-1, Hom/Mel-40, PRAME, p53, H-Ras, HER-2/neu, E2A-PRL, H4-RET, IGH-IGK, MYL-RAR, human papillomavirus (HPV) antigens E6 and E7, TSP-180, MAGE-4, MAGE-5, MAGE-6, pl85erbB2, pl80erbB-3, c-met, nm-23Hl, PSA, TAG-72-4, CA 19-9, CA 72-4, CAM 17.1, NuMa, K-ras, 13-Catenin, Mum-1, pl6, TAGE, PSMA, CT7, telomerase, 43-9F, 5T4, 791Tgp72, 13HCG, BCA225, BTAA, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90, TAAL6, TAG72, TLP, or TPS. Other tumor-associated and tumor-specific Ligand Binding Domains are known to those in the art.

A Ligand Binding Domain recognized by the DNL-CAR of the invention, or by the tagged-DNL used in the tagged-DNL/CAR-T system of the invention, may preferably be a Ligand Binding Domain not considered to be a TSA or a TAA, but which is nevertheless associated with tumor cells, or damage caused by a tumor. For example, the Ligand Binding Domain is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in other specific embodiments, the Ligand Binding Domain is a hypoxia-associated factor, e.g., HIF-1a, HIF-Iβ, HIF-2a, HIF-2p, HIF-3a, or HIF-3p. Tumors can also cause localized damage to normal tissue, causing the release of molecules known as damage associated molecular pattern molecules (DAMPs; also known as alarmins). Therefore, the Ligand Binding Domain may preferably be a DAMP, e.g., a heat shock protein, chromatin-associated protein high mobility group box 1 (HMGB1), S100A8 (MRP8, calgranulin A), S100A9 (MRP 14, calgranulin B), serum amyloid A (SAA), or can be a deoxyribonucleic acid, adenosine triphosphate, uric acid, or heparin sulfate.

The DNL-CAR of the invention, or the tagged-DNL used in the tagged-DNL/CAR-T system of the invention, may also target intracellular tumor associated Ligand Binding Domains such as HA-1, survivin, WT1, and p53. This can be achieved by a DNL-CAR of the invention expressed on a universal T cell that recognizes the processed peptide described from the intracellular tumor associated Ligand Binding Domain in the context of HLA. This can also be achieved with the tagged-DNL/CAR-T system of the invention by using a tagged-DNL that recognizes the processed peptide described from the intracellular tumor associated Ligand Binding Domain in the context of HLA. In addition, the universal T cell may be genetically modified to express a T-cell receptor pairing that recognizes the intracellular processed tumor associated Ligand Binding Domain in the context of HLA.

Preferably, the extracellular DNL domain of the DNL-CARs, or the extracellular domain of the CARs used in the tagged-DNL/CAR-T system described herein, is joined to the transmembrane domain of the polypeptide by a linker, spacer or hinge polypeptide sequence such as a sequence from CD28; a sequence from CTLA4; a CH2CH3 hinge region of an IgG1; or any other suitable linker that imparts flexibility and other desired features to the DNL-CAR or CAR of the tagged-DNL/CAR-T system.

A DNL-CAR, a tagged-DNL, and a CAR used in the tagged-DNL/CAR-T system according to the present invention can be produced by any means known in the art, though preferably it is produced using recombinant DNA techniques. A nucleic acid sequence encoding the several regions of the DNL-CAR, tagged-DNL, or CAR used in the tagged-DNL/CAR-T system can be prepared and assembled into a complete coding sequence by standard techniques of molecular cloning. The resulting coding region can be inserted into an expression vector and used to transform a suitable expression host allogeneic T-cell line. As used herein, a nucleic acid construct or nucleic acid sequence or polynucleotide is intended to mean a DNA molecule that can be transformed or introduced into a T cell and be transcribed and translated to produce a product (e.g., a DNL-CAR). Expression vectors are well known for expressing nucleic acids in a variety of different organisms, including mammalian cells, insect cells, bacteria and eukaryotic microorganisms such as yeasts. All such expression vectors are well known to those skilled in the art and the use of expression vectors to express the nucleic acid sequence is a standard technique well known to those skilled in the art.

Provided herein are nucleic acid sequences (polynucleotides) that encode one or more of the DNL-CARs of the invention, one or more of the tagged-DNLs of the invention, or one or more of the CARs used in the tagged-DNL/CAR-T system. The polynucleotides may be contained within any polynucleotide vector suitable for the transformation of immune cells, e.g., T lymphocytes. For example, T lymphocytes may be transformed using synthetic vectors, lentiviral or retroviral vectors, autonomously replicating plasmids, a virus (e.g., a retrovirus, lentivirus, adenovirus, or herpes virus), or the like, containing polynucleotides encoding the first and second polypeptides (e.g., chimeric receptors). Lentiviral vectors suitable for transformation of T lymphocytes include, but are not limited to, e.g., the lentiviral vectors described in U.S. Pat. Nos. 5,994,136; 6,165,782; 6,428,953; 7,083,981; and 7,250,299, the disclosures of which are hereby incorporated by reference in their entireties. HIV vectors suitable for transformation of T lymphocytes include, but are not limited to, e.g., the vectors described in U.S. Pat. No. 5,665,577, the disclosure of which is hereby incorporated by reference in its entirety.

Nucleic acids useful in the production of DNL-CARs, a tagged-DNL, or a CAR used in the tagged-DNL/CAR-T system include DNA, RNA, or nucleic acid analogs. Nucleic acid analogs can be modified at the base moiety, sugar moiety, or phosphate backbone, and can include deoxyuridine substitution for deoxythymidine, 5-methyl-2′-deoxycytidine or 5-bromo-2′-deoxycytidine substitution for deoxycytidine. Modifications of the sugar moiety can include modification of the 2′ hydroxyl of the ribose sugar to form 2′-0-methyl or 2′-0-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7: 187-195; and Hyrup et al. (1996) Bioorgan. Med. Chain. 4:5-23. In addition, the deoxyphosphate backbone can be replaced with, for example, a phosphorothioate or phosphorodithioate backbone, a phosphoroamidite, or an alkyl phosphotriester backbone.

Also provided herein are immune cells, e.g. T lymphocytes, comprising or expressing the DNL-CARs of the invention or the CARs used in the tagged-DNL/CAR-T system of the invention. The T lymphocytes provided herein may be naive T lymphocytes or MHC-restricted T lymphocytes. In certain embodiments, the T lymphocytes provided herein are tumor infiltrating lymphocytes (TILs). Preferably, the T lymphocytes provided herein have been isolated from a tumor biopsy, or have been expanded from T lymphocytes isolated from a tumor biopsy. Preferably, the T lymphocytes provided herein have been isolated from, or expanded from, T lymphocytes expanded from, peripheral blood, cord blood, or lymph.

The immune cells provided herein, comprise a DNL-CAR of the invention, e.g., DNL-CAR expressing T lymphocytes, or comprise a CAR used in the tagged-DNL/CAR-T system of the invention, may preferably be autologous to an individual to whom the modified T lymphocytes are to be administered. The DNL-CAR T lymphocytes or the CAR T lymphocytes used in the tagged-DNL/CAR-T system provided herein may preferably be allogeneic to an individual to whom the DNL-CAR T lymphocytes or the CAR T lymphocytes used in the tagged-DNL/CAR-T system are to be administered. Where allogeneic T lymphocytes are used to prepare modified T lymphocytes, T lymphocytes can be selected that will reduce the possibility of graft-versus-host disease (GVHD) in the individual. For example, virus-specific T lymphocytes can be selected for preparation of modified T lymphocytes; such lymphocytes will be expected to have a greatly reduced native capacity to bind to, and thus become activated by, any recipient Ligand Binding Domains. Recipient-mediated rejection of allogeneic T lymphocytes can be reduced by co-administration to the host of one or more immunosuppressive agents, e.g., cyclosporine, tacrolimus, sirolimus, cyclophosphamide, or the like.

T lymphocytes may preferably be obtained from an individual, optionally expanded, and then transformed with a polynucleotide encoding a DNL-CAR or a CAR used in the tagged-DNL/CAR-T system described herein, and optionally expanded. T lymphocytes may preferably be obtained from an individual, optionally expanded, and then transformed with a DNL-CAR of the invention or a CAR used in the tagged-DNL/CAR-T system invention and optionally expanded at least one more time. Cells containing the polynucleotides may be selected using a selectable marker.

The DNL-CAR T lymphocytes of the invention or the CAR T lymphocytes used in the tagged-DNL/CAR-T system of the invention may desirably express or comprise native TCR proteins, e.g., TCR-α and TCR-β that are capable of forming native TCR complexes. Either or both of the native genes encoding TCR-α and TCR-β in the DNL-CAR T lymphocytes can desirably be modified to be non-functional, e.g., a portion or all are deleted, a mutation is inserted, etc.

The signaling motifs of the transmembrane domain of the DNL-CARs or of the CARs used in the tagged-DNL/CAR-T system can desirably be used to promote proliferation and expansion of the modified T lymphocytes described herein. For example, unmodified T lymphocytes, and T lymphocytes comprising a DNL-CAR, or comprising a CAR used in the tagged-DNL/CAR-T system, comprising a CD3ζ signaling domain and a CD28 co-stimulatory domain can be expanded using antibodies to CD3 and CD28, e.g., antibodies attached to beads, or to the surface of a cell culture plate; see, e.g., U.S. Pat. Nos. 5,948,893; 6,534,055; 6,352,694; 6,692,964; 6,887,466; and 6,905,681. The DNL-CAR T lymphocytes or the CAR T lymphocytes used in the tagged-DNL/CAR-T system can desirably be used to promote selective expansion of T lymphocytes expressing the polypeptide. For example, when the Ligand Binding Domain is a TSA, T lymphocytes comprising the polypeptide cultured in the presence of the TSA, e.g., a soluble form of the TSA, result in increased proliferation as compared to culturing in the absence of the TSA.

The DNL-CAR T lymphocytes or the CAR T lymphocytes used in the tagged-DNL/CAR-T system can optionally comprise a “suicide gene” or “safety switch” that enables killing of all or substantially all of the T lymphocytes when desired. For example, the DNL-CAR T lymphocytes, or the CAR T lymphocytes used in the tagged-DNL/CAR-T system, can desirably comprise an HSV thymidine kinase gene (HSV-TK), which causes death of the modified T lymphocytes upon contact with gancyclovir.

The DNL-CAR T lymphocytes, or the CAR T lymphocytes used in the tagged-DNL/CAR-T system, may also desirably express or comprise an inducible caspase, e.g., an inducible caspase 9 (icaspase9), e.g., a fusion protein between caspase 9 and human FK506 binding protein allowing for dimerization using a specific small molecule pharmaceutical. See Straathof et al., Blood 105(11):4247-4254 (2005).

The DNL-CAR T lymphocytes of the invention, or the CAR T lymphocytes used in the tagged-DNL/CAR-T system of the invention, can preferably be used to treat an subject having one or more types of cells desired to be targeted by T lymphocytes, e.g., one or more types of cells to be killed. As used herein, the term “subject” or “patient” refers to any organism to which a composition in accordance with the invention may be administered, e.g., for experimental, diagnostic, prophylactic, and/or therapeutic purposes. Typical subjects include animals {e.g., mammals such as mice, rats, rabbits, non-human primates, and humans) and/or plants. In certain embodiments, the cells to be killed are cancer cells, e.g., tumor cells. Preferably, the cancer cells are cells of a solid tumor. Preferably, the cells are cells of a lymphoma, a lung cancer, a breast cancer, a prostate cancer, an adrenocortical carcinoma, a thyroid carcinoma, a nasopharyngeal carcinoma, a melanoma, e.g., a malignant melanoma, a skin carcinoma, a colorectal carcinoma, a desmoid tumor, a desmoplastic small round cell tumor, an endocrine tumor, an Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipoma, or the like. In more specific embodiments, said lymphoma can be chronic lymphocytic leukemia (small lymphocytic lymphoma), B-cell prolymphocytic leukemia, lymphoplasmacytic lymphoma, Waldenstrom macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, or a non-Hodgkin lymphoma.

Efficacy of the DNL-CAR T lymphocytes of the invention, or the CAR T lymphocytes used in the tagged-DNL/CAR-T system of the invention described herein, after administration to an individual having a disease or disorder remediable by T lymphocytes, e.g., an individual having cancer, can desirably be assessed by one or more criteria, specific to the particular disease or disorder, known to those of ordinary skill in the art, to be indicative of progress of the disease or disorder. Generally, administration of the DNL-CAR T lymphocytes of the invention, or the CAR T lymphocytes used in the tagged-DNL/CAR-T system of the invention, described herein to such an individual is effective when one or more of said criteria detectably, e.g., significantly, moves from a disease state value or range to, or towards, a normal value or range.

Desirably an effective amount or sufficient number of the DNL-CAR T lymphocytes, or CAR T lymphocytes used in the tagged-DNL/CAR-T system of the invention, is present in the composition and introduced into the subject such that long-term, specific, anti-tumor responses are established to reduce the size of a tumor or eliminate tumor growth or regrowth than would otherwise result in the absence of such treatment. Desirably, the amount of DNL-CAR T lymphocytes reintroduced, or the amount of CAR T lymphocytes (of the tagged-DNL/CAR-T system) reintroduced, into the subject causes a 10%, 20%, 30%, 40%, 50%, 60%, 70%, 80%, 90%, 95%, 98%, or 100% decrease in tumor size when compared to otherwise same conditions wherein the transduced T cells are not present.

Accordingly, the amount of DNL-CAR T lymphocytes administered, or the amount of CAR T lymphocytes (of the tagged-DNL/CAR-T system) administered, should take into account the route of administration and should be such that a sufficient number of DNL-CAR T lymphocytes or CAR T lymphocytes of the tagged-DNL/CAR-T system will be introduced so as to achieve the desired therapeutic response. Furthermore, the amounts of each active agent included in the compositions described herein (e.g., the amount per each cell to be contacted or the amount per certain body weight) can vary in different applications. In general, the concentration of DNL-CAR T lymphocytes, or CAR T lymphocytes of the tagged-DNL/CAR-T system, desirably should be sufficient to provide in the subject being treated at least from about 1^{χ}10⁶ to about 1×10⁹ DNL-CAR T lymphocytes or at least from about 1^{χ}10⁶ to about 1×10⁹ CAR T lymphocytes of the tagged-DNL/CAR-T system, even more desirably, from about 1×10⁷ to about 5×10⁸ DNL-CAR T lymphocytes, or from about 1×10⁷ to about 5×10⁸ CAR T lymphocytes of the tagged-DNL/CAR-T system, although any suitable amount can be utilized either above, e.g., greater than 5^{χ}10⁸ cells, or below, e.g., less than 1^{χ}10⁷ cells. The dosing schedule can be based on well-established cell-based therapies (see, e.g., Topalian and Rosenberg, 1987; U.S. Pat. No. 4,690,915), or an alternate continuous infusion strategy can be employed.

These values provide general guidance of the range of DNL-CAR T lymphocytes or CAR T lymphocytes of the tagged-DNL/CAR-T system to be utilized by the practitioner upon optimizing the method of the present invention for practice of the invention. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on inter-individual differences in pharmacokinetics, drug disposition, and metabolism. One skilled in the art readily can make any necessary adjustments in accordance with the exigencies of the particular situation.

The DNL-CAR T lymphocytes of the invention or CAR T lymphocytes of the tagged-DNL/CAR-T system of the invention described herein may be formulated in any pharmaceutically-acceptable solution, preferably a solution suitable for the delivery of living cells, e.g., saline solution (such as Ringer's solution), gelatins, carbohydrates (e.g., lactose, amylose, starch, or the like), fatty acid esters, hydroxymethylcellulose, polyvinyl pyrolidine, etc. Such preparations are preferably sterilized prior to addition of the DNL-CAR T lymphocytes or the CAR T lymphocytes of the tagged-DNL/CAR-T system of the invention, and may be mixed with auxiliary agents such as lubricants, preservatives, stabilizers, emulsifiers, salts for influencing osmotic pressure, buffers, and coloring. Pharmaceutical carriers suitable for use in formulating the DNL-CAR T lymphocytes of the invention or CAR T lymphocytes of the tagged-DNL/CAR-T system of the invention are known in the art and are described, for example, in WO 96/05309.

### Tagged DNL-Antigenic Fusion Proteins

The tagged DNL-antigenic fusion proteins of the invention may be used in place of, or in addition to the tagged DNL molecules of the tagged-DNL/CAR-T systems described herein. However, the presence of an antigenic protein fused to the tagged DNL eliminates the need to provide CAR-T cells and instead relies on recruitment of the patient's existing immune system to provide immune cells capable of neutralizing the cells that have been tagged with the tagged DNL portion of the fusion protein molecule.

Preferably the tagged DNL portion of the tagged DNL-antigen fusion protein includes all the features of the tagged-DNL molecule described herein. The tagged DNL-antigen fusion protein may be produced by recombinant nucleic acid techniques as described herein and as are known in the art for the production of fusion proteins.

Preferably the antigen portion of the tagged DNA is an antigen known to promote a non-life-threatening immune response in a patient. Preferably, such antigens include environmental antigens that cause non-life-threatening allergies in humans and other mammals including but not limited to: allergenic proteins associated with animals such as cat dander (e.g. Fel d 1), house dust mites (e.g. Der p 1 and Der p 2). Other environmental antigens include, but are not limited to allergenic proteins associated with pollens originating from trees, grasses and weeds and molds. Preferably, such antigens used herein include common protein antigens used to vaccinate patients against pathogens including, but not limited to: immunogenic protein fragments from: diphtheria, tetanus, pertussis, polio (IPV), measles, mumps, rubella, chickenpox Hepatitis A, b and H, influenzae and pneumococcal.

For example, the measles virus (MV) hemagglutinin protein (PH) is antigenically stable (Tahara et al., Viruses 2016, 8:216; doi:10.3390/v8080216)). The H protein is responsible for receptor binding, and is the main target of neutralizing antibodies. Human sera from vaccinated individuals and measles patients neutralized all MV strains with similar efficiencies, regardless of the N-linked sugar modification or mutations at these epitopes. The structure of the protein is well known and a tagged DNL-MVPH fusion that maintains the binding properties of the tagged DNL and the antigenic epitopes of the MVPH can be readily constructed and the tagged-DNL antigenic fusion protein produced in E. coli or another suitable production host (Bacillus, yeast, insect cells, etc.) as described herein.

Preferably, the invention provides a fusion protein comprising a tagged-DNL fused to an antigen wherein the antigen promotes a non-life-threatening immune response in a patient. Preferably the antigen is an environmental antigen. Preferably, the antigen is an antigen associated with a vaccine against a pathogen. Preferably, the vaccine is selected from the group of vaccines consisting of: diphtheria, tetanus, pertussis, polio (IPV), measles, mumps, rubella, chickenpox Hepatitis A, b and H, influenzae and pneumococcal.

Preferably, the tagged-DNL of the tagged-DNL-antigen fusion protein is a Pan-HER antagonist DNL. Preferably, the tagged-DNL is an IGF-I receptor antagonist DNL. Preferably, the tagged-DNL is a prolactin receptor antagonist DNL. Preferably the tagged-DNL is Human Growth Hormone (hGH) receptor antagonist DNL.

Preferably, the tag of the tagged-DNL-antigen fusion protein is selected from polyethylene glycol (PEG), FITC, strepaviidin, biotin, dinitrophenol, peridinin chlorophyll protein complex, green fluorescent protein, phycoerythrin, horse radish peroxidase, palmitolyation, nitrosylation, alkaline phosphatase, glucose oxidase and maltose binding protein.

Preferably, the DNL of the tagged-DNL-fusion protein is a ligand for a tumor-associated Ligand Binding Domain or tumor-specific Ligand Binding Domain, wherein the

DNL is selected from epidermal growth factor receptor (EGFR), HER2, HER3, HER4, IGF-IR, other tyrosine kinase receptors, other G-protein coupled receptors, prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD19, CD22, CD27, CD30, CD34, CD45, CD70, CD99, CD117, EGFRvIII (epidermal growth factor variant III), mesothelin, PAP (prostatic acid phosphatase), prostein, TARP (T cell receptor gamma alternate reading frame protein), Trp-p8, STEAP1 (six-transmembrane epithelial antigen of the prostate 1), chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-1), myo-Dl, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, and an abnormal p53 protein.

### Exemplary DNL-CAR T-Cell System Targeting EGFR

Certain classes of DNL can interact with more than one target receptor on the surface of the target cell. For example, the Pan-HER DNL reported in U.S. Pat. No. 7,557,181 is a re-engineered variant of EGF that binds to all of the ligand-dependent members of the HER family but does not stimulate signal transduction and phosphorylation. It inhibits the binding of authentic ligand. The Pan-HER DNL described in U.S. Pat. No. 7,557,181 has been shown to inhibit the activation of the Human Epidermal Receptor (HER) family of Receptor Tyrosine Kinases (RTK), the best studied and possibly the most important family of RTKs in cancer. The Pan-HER DNL is a small, readily made and highly stable protein. It is an engineered hybrid of Epidermal Growth Factor (EGF) and Heregulin (HRG) that interferes with the activation of the entire HER family of cellular receptors, including the important HER3 receptor.

Dysfunction of the HER system has been implicated in the progression of many forms of cancer, and many patients who survive initial rounds of HER-targeting therapeutics subsequently develop resistant tumors. Recent reports have highlighted the central importance of HER3 in tumor cell proliferation and demonstrated that traditional small molecule kinase inhibitors are irrelevant against this target, since it is not a kinase but rather the substrate for other kinases. In addition, HER3 has been shown to play a role in the development of resistance to HER-targeting drugs (Kol, A 2014 Pharmacol Ther. 2014 July;143(1):1-11).

There is no naturally occurring human growth factor (ligand) with affinity for EGFR, HER3 and HER4. There some engineered variants (such as T1E) that are Pan-HER agonists. T1E binds to EGFR, HER3 and HER4 with affinities comparable to the authentic cognate ligands, and stimulates EGF- or heregulin-dependent cell lines. This can be sharply contrasted with the Pan-HER DNL, which binds to all three ligand-dependent HER receptors, but does not stimulate receptor dimerization or activation.

Typically, HER ligands (and HER-targeting monoclonal antibodies) bind to their cognate receptors and stimulate receptor dimerization, followed by rapid internalization. On the contrary, the Pan-HER DNL binds to its cognate receptor but does not stimulate the subsequent cellular events that lead to internalization and cellular stimulation.

In recent years, it has been observed that tumor cells can develop resistance to HER-targeting therapeutics. One important drug resistance mechanism is the recruitment of other members of the HER axis or other receptors altogether, to take over for the particular receptor that has been inhibited. Lee-Hoeflich et al. (Lee-Hoeflich S T et al. 2008. A central role for HER3 in HER2-amplified breast cancer: implications for targeted therapy. Cancer Res. 68, 5878-876) have noted the central role of HER3 in acquired resistance to trastuzumab. These mechanisms have been recently reviewed (Chong, C R and P A Janne. 2013. The quest to overcome resistance to EGFR-targeted therapies in cancer. Nature Medicine 19(11)1389-1400). The importance of receptor cross talk and the central role of HER3 in HER2-amplified breast cancer are becoming increasing well-recognized (Ma J, et al. 2014. Targeting of ErbB3 receptor to overcome resistance in cancer treatment. Molecular Cancer 13:105). Consequently, there is an opportunity to interfere with the growth and survival of HER-ligand dependent tumors with a compound that exhibits a unique mechanism of action compared to antibody-based CAR T cells or CAR T Cells based on cell receptor antagonist.

A DNL-CAR T cell system of the invention using Pan-HER DNL would be superior to one using the active form of EGF, or one that uses a hybrid ligand, such as the T1E28+ system reported by Davies et al. (Davies M D et al. 2012. Mol Med 18:565-576) since in many tumor cells more than one of the HER receptors is expressed and the combination is important for cell proliferation and progression of the disease. For example, HER2/HER3 heterodimers are considered among the most powerful in the progression of some tumors.

In contrast, the T1E28+CAR reported uses a variant hybrid of transforming growth factor (TGF)-α and EGF (Wingens M. et al. 2006. Biochemistry 45:4703-4710) that binds to EGFR and HER3 and activates signal transduction and cell growth. A second limitation of molecules like the (TGF)-α/EGF protein in T1E28+ is that the targeting moiety (the fused ligand) is itself an agonist of receptor that it is targeting. This protein binds to the receptor and may cause receptor activation. The close proximity of the T cell may prevent this, but the unintended release of CAR ectodomain proteins into the extracellular medium are much more likely to bind and activate their cognate receptor which would be undesirable. There are many reports of shedding of the ectodomains of cellular receptors into the extracellular medium (Hayashida K, 2010. Amnat Rec (Hoboken) 293(6):925-37). Such shedding of agonist-based ectodomain would lead to unwanted activation of the target receptor. However, in contrast, shedding of the DNL in accordance with the present invention would lead to antagonism of receptor activation and may add to the anti-tumor activity of the immunotherapy.

The previously described Pan-HER DNL does not bind to HER2 but interferes with the activation of HER2/HER3 heterodimers, which are very important in the progression of many cancers and the development of resistance to HER-targeting drugs. The HER family of receptors includes four closely related members consisting of EGFR (HER1), HER2, HER3 and HER4. EGFR and HER2 are the targets for several FDA-approved molecular targeting agents, including cetuximab, panitumumab, erlotinib, gefitinib, trastuzumab and lapatinib. Despite significant clinical success, these drugs have not been nearly as effective in the clinic as first hoped and anticipated. This is due in part to the unexpected complexity of the “HER axis” and the ability of tumor cells to acquire resistance to these agents. In particular the central role of HER3 in the progression of cancer has only been recently appreciated.

A Pan-HER CAR T cell system of the invention takes advantage of the strong and specific binding properties of the DNL and its antagonist properties to direct the T cell to the appropriate target tumor cells and not stimulate signal transduction. In general, Pan-HER antagonists has been shown to be effective (Tao et al. 2014. Science Signaling 7(318):1-9). The PAN-HER CAR T cell system of the invention is directed against tumor cells that over express EGFR, HER2 or HER3, and in particular tumor cells that express more than one of the receptors, as is frequently the case in tumor cell lines that have developed resistance to therapeutics targeted at these receptors individually.

In one case, the Pan-HER DNL is derived from EGF and heregulin. It binds to all of the ligand-dependent members of the HER axis (EGFR, HER3 and HER4). Unlike traditional small molecule tyrosine kinase inhibitors (TKIs) that directly interfere with phosphorylation, or the blockade action of monoclonal antibodies, the molecule takes aim at another central feature of HER biology, the necessary binding of EGFR, HER3 and HER4 to their cognate ligands for activation. This engineered ligand has a new phenotype and an entirely different mechanism of action for interfering with HER-dependent tumor cell growth and proliferation.

Exemplary DNL-CAR T-Cell System Targeting IGF-1 and IGF-II

Another example of a DNL CAR T system utilizes a DNL created to target tumors that over express IGF-I and IGF-II. In this case, the DNL is a re-engineered variant of IGF-I that binds to IGF-I and IGF-II but does not initiate signal transduction (WO2008005985-Insulin-like growth factor-1 receptor antagonists).

Exemplary DNL-CAR T-Cell System Targeting hGH

Another example of the DNL-CAR T lymphocyte is based on a fusion of the TCR endodomain and a variant of Human Growth Factor. The PEGylated version of one such variant (Pegvisomant), has been successfully used to inhibit hGHR in patients with an abnormal abundance of hGH. (This over abundance leads to the disease acromegaly). It has been recently shown that hGHR is overexpressed in some pancreatic tumor cells (Subramani R, et al. 2014). Growth hormone receptor inhibition decreases the growth and metastasis of pancreatic ductal adenocarcinoma, Experimental and Molecular medicine 46:e117). A fusion of TCR to the cognate ligand could lead T cells to these tumors and result in their death.

Exemplary DNL-CAR T-Cell System Targeting Prolactin

Another example of a DNL-CAR T cell utilizes a DNL based on Prolactin (PRL). PRL is a neuroendocrine hormone involved in the development and differentiation of the mammary gland and ovary, and its biological activities are mediated by a specific membrane receptor, the PRL receptor (PLRL). Much like human growth hormone, a single amino acid substitution (G129R) turns the agonist into an antagonist. This variant prevents the dimerization of the prolactin receptor (PRLR), disrupting various signaling pathways suppresses tumor cell growth.

Exemplary DNL-CAR T-Cell System Targeting Other Proteins

Other ligands can also be turned in DNL, making new DNL-CAR T cells that target other cellular receptors implicated in the progression of disease, including interferon receptors, hGH receptors, VEGF receptors, NGF receptors, TNF receptors, G-protein coupled receptors and any other receptor pathway known to operate, be triggered or function via polypeptide ligand binding.

Exemplary Tagged DNL/CAR T-Cell System

Currently, CAR T cells can only be aimed at a single target at a time since the CAR has only a single antibody-based antigen recognition and binding region. This is important because development of basic CAR T therapy is a laborious and time-consuming (>2-3 months) process. In order to achieve a sufficient number of tumor-reactive T cells, T cells must be initially removed from the patient, transformed with the new DNA, enriched and amplified, and then reintroduced into the patient. It would be useful if a single population of CAR T cells could target more than one target on the surface of the cell, since generating multiple rounds of CART cells is impractical.

An approach has been recently proposed that addresses these limitations. It has been recently shown that CAR T cells can be directed in vivo to a particular target tissue and cell using an indirect “tagging” system (Abate-Daga and Davila, 2016 and Tamada et al, 2012). In these systems the Chimeric Antigen Receptor consists of a scFV domain from an anti-fluorescein isothiocyanate (FITC) monoclonal antibody, which is connected to the signaling motifs of CD28, 4-1 BB and CD3ζ (referred to as an “anti-FITC CAR”). This anti-FITC CAR can be directed to FITC-labeled antibodies which have themselves attached to particular antigens on the surface of target tumor cells. In this way “FITC-tagged” monoclonal antibodies can direct a generic anti-FITC CAR to different tumor targets.

US patent application 20160129109 discusses the use of other tags besides FITC, including strepaviidin, biotin, dinitrophenol, peridinin chlorophyll protein complex, green fluorescent protein, phycoerythrin, horse radish peroxidase, palmitolyation, nitrosylation, alkaline phosphatase, glucose oxidase and maltose binding protein (claim 4). It does not discuss the use of PEGylation as a method to tag the monoclonal. US 20160129109 also limits its claims to antibodies or “antigen-binding” proteins.

We have developed a novel way to use a Dominant Negative Ligand (DNL) as the targeting agent. A tagged version of a DNL that retains its antagonist activity is used in combination with a CAR as a new system for targeting pathological cells in situ. One embodiment of the invention comprises tagging a DNL with a substance that is recognized by a CAR. A second embodiment comprises genetically engineering T cells to express a CAR that recognizes and binds to the tag on the DNL. A further embodiment comprises treating a cancer patient using a tagged DNL and a CAR-T cell that recognizes and binds to the tag on the DNL.

Here we describe the successful modification (“tagging”) of a Pan-HER DNL as the agent that binds to a target tumor. The tagged Pan-HER DNL binds to its cognate HER receptor on a tumor. The Pan-HER DNL is a modified ligand of HER and is not an antibody, or derived from an antibody and is not an antigen-binding protein. Pan-HER DNL binds to a specific, defined binding site on the surface of HER2, HER3 and HER4. The Pan-DER DNL has the significant advantage over antibody-based tags of being a powerful tumor antagonist in its own right as it interferes with activation by authentic ligands.

EGF has been successfully PEGylated at its N-terminus (Lee, H and G. Park. 2002. Preparation and Characterization of Mono-PEGylated Epidermal Growth Factor: Evaluation of in vitro Biologic Activity. Pharmaceutical Research 19(6):845-851). However, to make effective DNLs, we PEGylated the DNL at the N-terminus (variant B5-2M3), and at the N-terminus and a residue in the interior of the protein (K26) known to be required for ligand binding at Domain I (Variant B5-3M2). It was not predictable that the Pan-HER DNL would retain binding and antagonist activity after PEGylation since we purposely incorporated a PEGylation site directly into the middle of a binding region (K26). It was reasonable to expect that this would ruin the properties of the Pan-HER DNL. Instead, the DNL retained binding activity, inhibited ligand dependent tumor cell growth, and inhibited internalization of authentic EGFR and HER3 ligands. This result was unexpected.

An additional advantage of using the PEG tag on a DNL is that the tag extends the serum halflife of the DNL. The serum half-life of the Pan-HER DNL is expected to be about the same as unmodified EGF or human growth factor (about 10 minutes). This may be acceptable or even desirable for some applications, where rapid clearing of the drug is beneficial. In most of the anticipated applications, however, longer retention time in the body is desirable. We followed the successful example of Pegvisomant and increased the halflife of the protein through selective PEGylation (Clark et al.).

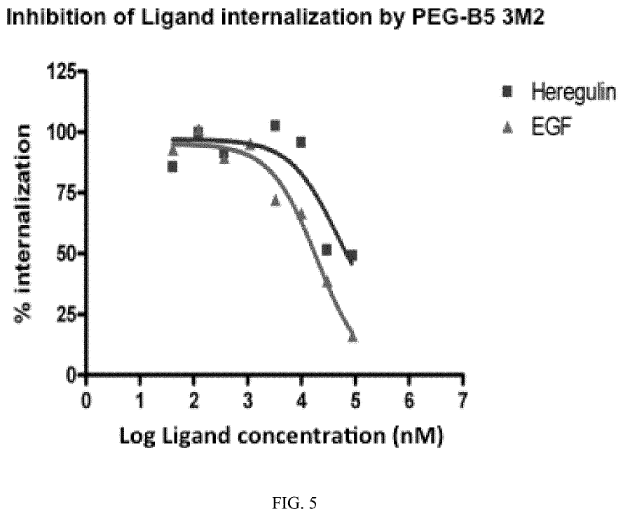

We found that Pan-HER DNL can be tagged with a small molecule (biotin) or a large molecule (polyethylene glycol, PEG) in such a fashion as to retain binding properties and the ability to block the binding of authentic ligands such as EGF or heregulin. The Pan-HER DNL B5 3M2 has two sites for biotinylation or PEGylation, the N terminus and a single internal lysine (K26). We have shown that B5-3M2 can be easily and reproducibly biotinylated or PEGylated at these sites and still retain its antagonist phenotype. In particular, the results show that PEGylated B5-3M2 inhibits internalization of the authentic ligand (EGF for EGFR, and Heregulin for HER3), just like the un-PEGylated protein, and is suitable for use as a target for a PEG-targeting CAR. (FIG. 5).

Tags can include other protein fusions (albumin, etc.) and other antigenic molecules that can be recognized by CAR-T cells or by the patient's own immune system.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

Example 1. Pan-HER DNL-CAR T therapeutic. Production of the Pan-HER DNL CAR T cells can be accomplished by first creating a fusion of the gene for the Pan-HER DNL protein and the genes for the CD28+CD3ζ endodomain.

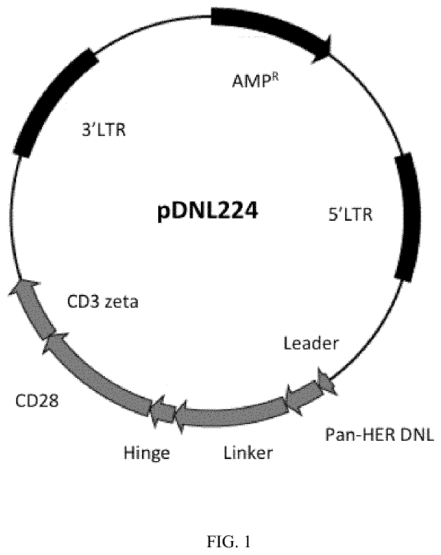

The ectodomain can be custom synthesized as a sequence-optimized DNA fragment that includes an appropriate leader sequence (such as the leader sequence of the human colony-stimulating factor-1 receptor) upstream of the Pan-HER DNL coding sequence such as those described in U.S. Pat. No. 7,557,181. The sequence may include a linker region to properly position the DNL to interact with its target Ligand Binding Domain. This sequence can then be subcloned in frame into a pSFG retroviral vector (such as those offered by Cell Biolabs, Inc. San Diego Calif.) containing a short hinge, CD28 transmembrane and signaling domain, and the signaling domain of the CD3ζ-chain. See, FIG. 1. Following amplification and transfection the resulting gene fusion can then be expressed in Human Embryonic Kidney 293 (“293”) cells. The 293T cell retroviral supernatant can then be used to transfect target T cells.

The following are the amino acid sequences of wild type EGF (SEQ ID NO: 1), T1E (modified EGF; SEQ ID NO: 2) and two HER-Targeting Dominant Negative Ligands of EGF useful in accordance with the invention: Pan-HER DNL B5-2M3 (SEQ ID NO: 3) and Pan-HER DNL B5-3M2 (SEQ ID NO: 4):

EGF
(SEQ ID NO: 1)
NSDSECPLSHDGYCLHDGVCMYIEALDKYACNCVVGYIGERCQYRDLKWW
ELR;
T1E
(SEQ ID NO: 2)
VVSHFNDCPLSHDGYCLHDGVCMYIEALDKYACNCVVGYIGERCQYRDLK
WWELR;
Pan-HER DNL B5-2M3
(SEQ ID NO: 3)
NSDSECPLSHDGYCLNDGVCMYTEALDEYACNCVVGYIGERCQYYDLL;
and
Pan-HER DNL B5-3M2
(SEQ ID NO: 4)
NSDSECPLSHDGYCLNDGVCMYTEAKDEYACNCVVGYIGERCQYYDLL.

Amino acids that are different from wild type EGF (SEQ ID NO: 1) are underlined. The Pan-HER DNL sequence B5-3M2 (EGF₁₋₄₈H16N, I23T, L26K, K28E, R45Y, K48L) of SEQ ID NO: 4 is one of many possible DNLs based on the ablation/binding optimization process to produce DNLs. It is significantly different from EGF (SEQ ID NO: 1) in that it is 5 amino acids shorter and has 6 different amino acids, is an antagonist rather than an agonist, and has Pan-HER binding specificity rather than just binding specificity for EGFR. The Pan-HER DNL is also significantly different from T1E (SEQ ID NO: 2) in that the Pan-HER DNL is 7 amino acids shorter, has 6 different amino acids, is an antagonist rather than an agonist, and has a different binding specificity as compared to T1E.

The gene sequences for the CD28 (NCBI/GENBANK Reference Sequence: NM_006139.3, available from OriGene, Inc. Rockville Md.) and CD3ζ (NCBI/GENBANK Reference Sequence: NM_198053 available from OriGene, Inc. Rockville Md.) endodomain may include codon optimized nucleic sequences for expression in the human T cells host. Other short sequences (e.g. for the hinge and linker regions) can be readily synthesized (Origen, Inc. Rockville Md.).

Human blood mononuclear cells are obtained and transfected with the retroviral supernatant. Transfected T cells can be maintained and propagated in an appropriate medium such as RPMI 1640+10% human AB serum (Sigma-Aldrich).

Surface expression of the Pan-HER DNL domain can be verified by flow cytometry, for example using a Coulter EPICS XL cytometer using goat anti-EGF antiserum (R&D Systems) followed by fluorescein isothiocyanate-conjugated rabbit-anti goat IgG (Dako).

The in vitro activity can be assessed using cell lines that express different levels of the HER family of receptors. Confluent monolayers of the target tumor cell lines can be co-cultivated in 24-well plates with approximate 10⁶ pan-HER DNL CAR T cells. After 3 days the cultures can be fixed and stained with crystal violet. Significant changes in the appearance of the plates (compared to controls) indicates that the T cells have affected the target tumor cells.

A panel of potential tumor cells lines can be assembled and their HER expression phenotype deduced using HER-specific antibodies and flow cytometry, similar to the approach taken by Davies et al. (2012). Alternatively, cell lines with known expression patterns of the HER receptors can be used, such as A431 (a human epidermal carcinoma line that over expresses EGFR which can be propagated in DMEM/F-12 media with 10% FBS), MDA-MB-453 (a human breast metastatic carcinoma cell line MDA-MB-453 that only expresses significant amounts of HER2 and HER3 which can be propagated in Liebovitz's L15 media with 10% FBS at ambient CO₂)), MDA-MB-175 (a human breast ductal carcinoma cell line that is resistant to the HER2-targeting monoclonal antibody trastuzumab trastuzumab (a potent inhibitor of HER2 dimerization) and can be propagated in Liebovitz's L15 media with 10% FBS at ambient CO₂), T47D (a human breast cancer cell line that expresses all four receptors and can be propagated in RPMI-1640 media with 10% FBS and 1% ITS-X supplement). A negative control cell line (such as SKBR2, which over expresses HER2 but not EGFR or HER3) can be used to demonstrate the specificity of the Pan-HER DNL to EGFR, HER3 and HER4 (but not HER2), and the lack of any general toxicity. All cell lines are grown at 37° C. and 5% CO₂ unless otherwise noted.

Example 2. IGF-I/II DNL-CAR T cells. Production of the IGTI/II DNL CAR T cells can be accomplished by first creating a fusion of the gene for an IGF-I/II Pan-HER DNL protein (such as those described in U.S Pat. Pub. 2009/0311783) and the genes for the CD28+CD3ζ endodomain. The ectodomain can be custom synthesized as a sequence-optimized DNA fragment that includes an appropriate leader sequence (such as the leader sequence of the human colony-stimulating factor-1 receptor) upstream of the IGF-I/II DNL coding sequence. The sequence may include a linker region to properly position the DNL to interact with its target Ligand Binding Domain. This sequence can then be subcloned in frame into a pSFG retroviral vector (such as those offered by Cell Biolabs, Inc. San Diego Calif.) containing a short hinge, CD28 transmembrane and signaling domain and the signaling domain of the CD3ζ-chain. Following amplification and transfection the resulting gene fusion can then be expressed in Human Embryonic Kidney 293 (“293T”) cells. The 293T cell retroviral supernatant can then be used to transfect target T cells.

Human blood mononuclear cells are obtained and transfected with the retroviral supernatant. Transfected T cells can be maintained and propagated in an appropriate medium such as RPMI 1640+10% human AB serum (Sigma-Aldrich).

Surface expression of the IGF-I/II domain can be verified by flow cytometry, for example using a Coulter EPICS XL cytometer using goat anti-IGF-I antiserum (R&D Systems) followed by fluorescein isothiocyanate-conjugated rabbit-anti goat IgG (Fisher Scientific).

The in vitro assessment of activity can be assessed using cell lines that express different levels of IGF-I, IGF-II and the Insulin Receptor. A panel of potential target and control tumor cells lines can be assembled and evaluated for their IGT-IR, IGF-IIR and IR expression phenotype deduced using specific antibodies and flow cytometry, similar to the approach taken by Davies et al. (2012). Confluent monolayers of the target tumor cell lines can be co-cultivated in 24-well plates with approximate 10⁶ IGF-I/II DNL CAR T cells. After 3 days the cultures can be fixed and stained with crystal violet. Significant changes in the appearance of the plates (compared to controls) indicates that the T cells have affected the target tumor cells.

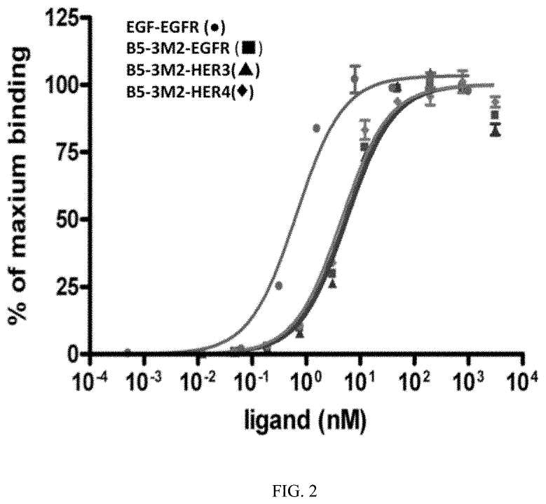

Example 3. Tagged (biotinyated) DNL retains activity. The Pan-HER DNL (B5-3M2) that has been derivatized at the N-terminus still bind to its cognate ligands. See FIG. 2.

Production and purification of the protein was carried out using standard methods. The E. coli production strain is grown in 400 ml Terrific Broth cultures in 2.8 liter Fernbach flasks at 37° C. Cultures are induced overnight with 0.2 mM IPTG, and supernatants collected by centrifugation. The protein is concentrated by ammonium sulfate precipitation and purified by Ni-NTA affinity chromatography. The His-tag is enzymatically removed, and the tag and un-cleaved material removed by a second Ni-NTA column. The protein is then further purified by gel filtration (FPLC) chromatography. Purified proteins are made up to 200 ug/ml in PBS, filter sterilized, and tested for endotoxin via chromogenic LAL test to insure safe levels (<5 EU/ml) for the subsequent animal studies.

Receptor affinities were measured using sandwich ELISA. Anti-Fc antibody (5 μg/ml in 50 mM carbonate buffer, pH 9.6, Jackson Immunoresearch) was pre-coated onto Nunc Maxisorb 96 well plates as capture antibody. Wells were blocked with 1% BSA in PBS (3.2 mM Na2HPO₄, 0.5 mM KH₂PO₄, 1.3 mM KCl, 135 mM NaCl, pH 7.4) for 2 hours at room temperature. Blocked plates were coated with the receptor of interest by addition of 100 μl of the target recombinant human HER/Fc chimera—EGFR, HER3 or HER4 (R&D systems) at 0.5 μg/ml in TBST-BSA (TBS with 0.05% Tween 20, 0.1% BSA, pH 7.2-7.4) to each well. Serial dilutions of labeled ligands were prepared and assayed on 96-well plates previously prepared with the receptors of interest. Ligand dilutions were applied to receptor-coated plates, which were then incubated for 2 hours at room temperature. Following incubation, wells were washed five times with 0.05% Tween 20 in PBS (PBST). Washed plates were probed with Streptavidin conjugated horseradish peroxidase and developed with 100 μl Tetramethylbenzidine (TMB) substrate solution (Pierce) to detect the bound biotinylated ligand (according to the manufacturer's directions). Bound ligand was detected by measurement of absorbance at 450 nm.

### Example 4 Tagged (PEGylated) DNL Retains Activity

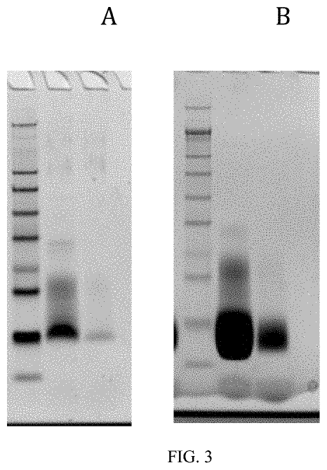

PEGylation reagents were obtained from Dow Pharma (Midland, Mich.). For the preliminary experiments, we used para-Nitrophenyloxycarbonyl-PEG derivatives for the PEGylation studies. “PNP-PEG” is a well established starting material and generates a higher percentage of conjugated and stable product compared to other linkers (such as NHS esters). Production conditions: borate-phosphate buffer (pH 8.0-8.3), room temperature with gentle stirring, overnight). The PEGylated DNL was be purified by dialysis (MW cutoff=10,000) in Tris buffer (Tris-HCl, 5 mM, pH 8.0). This removes un-reacted PEG, un-reacted EGF and other chemical reagents. The result of the purification is shown in FIG. 3.

### Example 5 Tagged (PEGylated) Pan-HER DNL Inhibits Ligand-Dependent Cell Proliferation

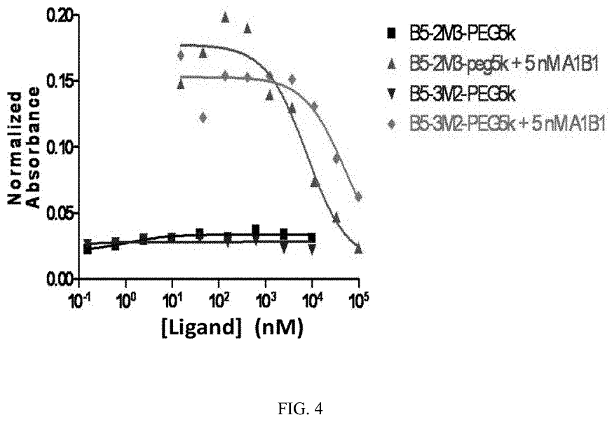

T47D, a human breast cancer cell line, was propagated in RPMI-1640 media with 10% FBS and 1% ITS-X supplement. Cells were incubated with or without the Pan-HER Agonist “A1B1” (which stimulates growth of the cells) and with increasing concentrations of the PEGylated DNL B5-2M3 (PEGylated only at one N-terminal site) or the B5 3M2 candidate (PEGylated at both the one N-terminal site and at a single internal site). The results are illustrated in FIG. 4. Cultures without the agonist A1B1 failed to grow. In cultures with the agonist, cell growth (as measured by absorbance) decreased as the concentration of PEGylated B5-2M3 or PEGylated B5-3M2 increased. This demonstrates the antagonist activity of the tagged DNLs.

### Example 6 PEGylated DNL Inhibits Internalization of Authentic Ligands

T47D, a human breast cancer cell line, was propagated in RPMI-1640 media with 10% FBS and 1% ITS-X supplement. Cells were grown to 70-80% confluence then serum starved overnight. The cells were incubated with Fluor 647-labelled ligands AF647-EGF or AF647-Heregulin at 4° C. for 30 min, washed 3 times with cold PBS, and then incubated at 37° C. for 30 min with increasing concentrations of PEGylated B5 3M2 (to allow internalization), and then placed on ice to stop the process. Cells were rinsed 3 times with cold PBS and subjected to an acid wash (0.2 M acetic acid and 0.5 M NaCl, pH 2.8) for 5 min followed by 3 washes with PBS to remove surface bound ligands. The cells were detached from tissue culture dishes with trypsin EDTA, washed, suspended in PBS with 2% fetal bovine serum and 0.01% sodium azide, and fixed by adding an equal volume of 4% formaldehyde/PBS.

Cells that had internalized ligands were separated from cells that had not and counted using Becton Dickinson FACSCalibur Flow Cytometer (BD Biosciences) at the University of Texas MD Anderson Cancer Center Flow Cytometry Core Facility. Data analysis was processed using FlowJo software (Tree Star Inc).

The results (FIG. 5) show that PEGylated B5 3M2 inhibits ligand-dependent internalization of the authentic ligands (EGF or heregulin) and is suitable for use as a target for a PEG-targeting CAR.

### Example 7 Tagged DNL—Measles Virus Fusion Protein H Fusions

Measles virus (MV) is an enveloped virus in the genus Morbillivirus of the family Paramyxoviridae and possesses two types of glycoprotein spikes, the hemagglutinin (H) and fusion (F) proteins, on the viral envelope. In the DNL-tag and DNL-tag fusions the immunogenic MV polypeptides can comprise any portion of these polypeptides that have at least one epitope capable of eliciting a protective immune response (cellular or humoral) against MV infection. Although neutralizing Abs directed against each of the viral envelope glycoproteins are elicited, H protein-specific Abs mainly account for the protection against MV infection. Target epitopes include the antigenic site vi, which is unrelated to receptor binding but probably involved in the formation of a higher-order H-F protein oligomeric structure. It is a major neutralizing epitope that is conserved among different genotype strains. In particular, amino acid sequences TYLVEKPNLSSKRSELSQLSMY (SEQ ID NO: 5) and TYLVEKPNL (SEQ ID NO: 6)-QLSMY (SEQ ID NO: 7) in the protein have been implicated in antigenicity. (Functional and structural characterization of neutralizing epitopes of measles virus hemagglutinin protein. M Tahara et al J Virol. 2013 Jan; 87(1): 666-675.) The DNA sequences coding for these amino acids can be incorporated into the n-terminal end of the Pan-HER DNL gene. This region has been shown to be unnecessary for ligand binding. The fusion protein can be produced in E coli or other bacterial or eukaryotic production systems.

The DNL-tagged fusion can also be synthesized by standard chemical methods, including synthesis by automated procedure. Immunogenic polypeptides or peptides can be isolated from authentic (attenuated) virus using standard methods. For example, host cells (such as baculovirus and mammalian cell lines) containing H or F or N protein immunogen-encoding nucleic acid expression constructs can be cultured to produce recombinant H or F or N protein immunogens, or fragments thereof (see, e.g., Piltz et al., Intl. J. Parasitol. 33:525 (2003). Crude immunogenic peptide may be further purified using preparative reverse phase chromatography. Other purification methods, such as partition chromatography, gel filtration, gel electrophoresis, ion-exchange chromatography, or other methods.

The immunogenicity of the DNL-tagged fusions can be evaluated with animal systems. For example BALB/c 10-week old female mice have been used for this application. Body weight and signs of toxicity (i.e., fur erection, hunched posture, oily skin, eye secretions, dehydration) are monitored every 2 days throughout the experiment. Fusions can be administered intranasally. Control groups receive the DNL- or DNL-tag without the fused immunogenic component. Standard serological methods can be used to verify the immunogenicity of the constructs.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention.

1. A dominant-negative ligand chimeric antigen receptor (DNL-CAR) comprising an intracellular activation domain, an optional costimulatory molecule domain, a transmembrane domain, an extracellular domain, and an optional linker between the transmembrane domain and the extracellular domain, wherein the extracellular domain comprises a dominant-negative ligand (DNL).

2. T Cells expressing the DNL-CAR of claim 1.

3. The DNL-CAR of claim 1, wherein the DNL is a Pan-HER antagonist DNL.

4. T Cells expressing the Pan-HER antagonist DNL-CAR of claim 3.

5. The DNL-CAR of claim 1, wherein the DNL is an IGF-I receptor antagonist DNL.

6. T Cells expressing the IGF-I receptor antagonist DNL-CAR of claim 5.

7. The DNL-CAR of claim 1, wherein the DNL is a prolactin receptor antagonist DNL.

8. T Cells expressing the prolactin receptor antagonist DNL of claim 7.

9. The DNL-CAR of claim 1, wherein the DNL is Human Growth Hormone (hGH) receptor antagonist DNL.

10. T cells expressing the hGH receptor antagonist DNL of claim 9.

11. A nucleic acid expressing a DNL-CAR of claim 1.

12. A plasmid comprising the nucleic acid of claim 11.

13. T cells comprising the plasmid of claim 12.

14. A pharmaceutical formulation comprising the T cells of claim 2.

15. A method of reducing the size of a tumor comprising contacting the tumor with a T cell of claim 2.

16-27. (canceled)

28. A fusion protein comprising a tagged-DNL fused to an antigen wherein the antigen promotes a non-life-threatening immune response in a patient.

29. The fusion protein claim 28, wherein the antigen is an environmental antigen.

30. The fusion protein of claim 28, wherein the antigen is an antigen associated with a vaccine against a pathogen.

31. The fusion protein of claim 30, wherein the vaccine is selected from the group of vaccines consisting of: diphtheria, tetanus, pertussis, polio (IPV), measles, mumps, rubella, chickenpox Hepatitis A, b and H, influenzae and pneumococcal.

32. The fusion protein of claim 28, wherein the tagged-DNL is a Pan-HER antagonist DNL.

33. The fusion protein of claim 28, wherein the tagged-DNL is an IGF-I receptor antagonist DNL.

34. The fusion protein of claim 28, wherein the tagged-DNL is a prolactin receptor antagonist DNL.

35. The fusion protein of claim 28, wherein the tagged-DNL is Human Growth Hormone (hGH) receptor antagonist DNL.

36. The fusion protein of claim 28, wherein the tag of the tagged-DNL is selected from polyethylene glycol (PEG), FITC, strepaviidin, biotin, dinitrophenol, peridinin chlorophyll protein complex, green fluorescent protein, phycoerythrin, horse radish peroxidase, palmitolyation, nitrosylation, alkaline phosphatase, glucose oxidase and maltose binding protein.

37. The fusion protein of claim 28, wherein the DNL of the tagged-DNL is a ligand for a tumor-associated Ligand Binding Domain or tumor-specific Ligand Binding Domain, wherein the DNL is selected from epidermal growth factor receptor (EGFR), HER2, HER3, HER4, IGF-IR, other tyrosine kinase receptors, other G-protein coupled receptors, prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD19, CD22, CD27, CD30, CD34, CD45, CD70, CD99, CD117, EGFRvIII (epidermal growth factor variant III), mesothelin, PAP (prostatic acid phosphatase), prostein, TARP (T cell receptor gamma alternate reading frame protein), Trp-p8, STEAP1 (six-transmembrane epithelial antigen of the prostate 1), chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-1), myo-Dl, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, and an abnormal p53 protein.
